(19) United States
(12) Patent Application Publication
(10) Pub. No.: US 2026/0191676 A1
(43) Pub. Date: Jul. 9, 2026

(54) Anti-Snoring Smart Pillow with Dynamic 3D Airbag System and Integrated Snoring Detection Integrated Snoring Detection

(71) Applicant: Mohamed Allaoui, Rorbas (CH)

(72) Inventor: Mohamed Allaoui, Rorbas (CH)

(21) Appl. No.: 19/013,558

(22) Filed: Jan. 8, 2025

(51) Int. Cl. A61F 5/56; A47G 9/00; A47G 9/10

(52) U.S. Cl. CPC A61F 5/56; A47G 9/1027; A47G 2009/003

(57) ABSTRACT

The invention is a smart anti-snoring pillow (SASP) that adjusts the user's head and neck position dynamically based on detected snoring patterns. Controlled via a mobile application, it uses a self-learning snoring detection system and advanced artificial intelligence and machine learning algorithms for accurate detection and customization. The pillow features a 3D airbag system that gently inflates and deflates within 10 seconds, incrementally adjusting angles between 20 and 45 degrees to align airways non-invasively. Its three-step operation starts with neck adjustment, followed by progressive airbag angle modifications if snoring persists. This approach improves breathing, enhances trachea ventilation, and reduces apnea risks linked to cardiovascular disease. The SASP's mobile integration allows for real-time adaptability, remote updates, and user-defined profiles (light, mild, or loud snorer). With a 98% snoring detection accuracy and self-improving capability, the scalable, cost-effective design addresses sleep technology gaps while ensuring comfort and minimal disruption for users.

### FIELD OF THE INVENTION

The present invention relates to a smart pillow design for reducing or preventing snoring and improving breathing during sleep phases. The invention integrates hardware and software components to monitor and adjust head and neck positions dynamically for enhanced sleep quality.

### BACKGROUND

Conventional sleep apnea treatments often involve approaches that, while effective in managing symptoms, can be uncomfortable, cumbersome, or intrusive for many patients. One of the most common treatments is the use of Continuous Positive Airway Pressure (CPAP) machines. These devices consist of a motorized unit that delivers pressurized air through a mask worn over the nose or mouth. While CPAP machines effectively prevent airway collapse, many users find them challenging to adapt to due to the bulkiness of the equipment, the sensation of pressurized air, and the need to wear a tightly-fitted mask throughout the night. Masks can cause skin irritation, pressure sores, and feelings of claustrophobia, while the machine's noise and airflow can disrupt sleep for both the user and their partner. Furthermore, the consistent maintenance required to keep the equipment clean and functional adds to the burden. Another common treatment option is the use of mandibular advancement devices, which are oral appliances designed to hold the lower jaw forward to keep the airway open. While these devices are less invasive than CPAP, they can cause significant discomfort, including jaw pain, stiffness, and soreness. Long-term use may lead to dental problems, such as tooth movement or misalignment, and can exacerbate temporomandibular joint disorders. For many users, the trade-off between mild relief and persistent discomfort makes adherence difficult.

Surgical interventions, such as uvulopalatopharyngoplasty, genioglossus advancement, or maxillomandibular advancement, are more permanent solutions but come with their own set of challenges. Surgery is invasive, requiring general anesthesia and an extended recovery period, during which patients may experience significant pain, swelling, and difficulty swallowing. The results of surgical procedures can vary widely, with some patients experiencing incomplete symptom relief or recurrence of apnea over time. Additionally, the irreversible nature of these interventions makes them a daunting choice for many. Even less invasive approaches, such as positional therapy or nasal dilators, often fail to provide adequate relief for moderate to severe cases of sleep apnea. These methods can feel restrictive, require constant monitoring to ensure compliance, and may not effectively address the underlying airway obstruction. Many patients also report frustration with trial-and-error methods to find the right combination of treatments, leading to dissatisfaction and reduced adherence. Developments in snoring pillow apparatus continue to evolve. In the United States, Patent No. US20180042408A1 disclosed an air-bag-lifting sleep pillow structure. United States Patent No. US20160286974A1 teaches of a snoring recognition and electric power adjustment. United States Patent No. US20210030583A1 disclosed an anti-snoring multiple height and slope pillow. Japan Patent No. JP2014138688A disclosed an automatic height adjustment sleeping pillow. Chinese Patent No. CN105902100A Pillow and method for detecting head position through acceleration sensors. Chinese Patent No. CN205094053U teaches of an intelligent snore relieving pillow. Chinese Patent No. CN218279231U disclosed an artificial intelligence sleep pillow. Chinese Patent No. CN114424877A and CN215271858U disclosed pillows with artificial intelligence assists sleep functions. Korean Patent No. KR20200070525A disclosed an AI pillow which has voice recognition function with using speakerphone. None of the prior art disclosed relies on air to adjust the head - rather they rely on mechanical operations such as rollers etc.

No patents were found that incorporated the functions of tilting the head first and the head second to an infinite number of angles. In addition to this disparity, none were found to have these features in combination with AI functions. What is needed is an inflatable pillow that adjusts the angle of the head dynamically offers a promising and user-friendly solution for managing sleep apnea and snoring. Unlike conventional treatments, this approach is non-invasive, comfortable, and integrates seamlessly into a user's nightly routine. By using adjustable air chambers, the pillow can gently modify the position of the head and neck in response to detected snoring or airway obstruction.

### SUMMARY OF THE INVENTION

The device herein disclosed and described provides a solution to the shortcomings in the prior art through the disclosure of a smart anti-snoring pillow that adjusts the user's head and neck position dynamically based on detected snoring patterns. An object of the SASP is to improved airway alignment automatically. The device detects snoring in real-time and inflates air bags to allow the user head to lean in at an angle that opens the airway using. Dynamic adjustments help maintain an open airway, reducing snoring and the risk of apnea episodes. Better head and neck positioning improves airflow and oxygen saturation, reduces strain on the cardiovascular system, and lowers risks associated with untreated sleep apnea. The device avoids the high costs associated with CPAP machines, surgeries, or multiple devices. The SASP requires less upkeep compared to CPAP machines or oral devices and is cheaper.

Another object of the SASP is to enhance comfort. The SASP eliminates the need for masks, oral appliances, or surgical interventions. Gentle, gradual adjustments of the device ensure the sleeper is not disturbed during operation.

Another object of the SASP is to allow users to personalize settings to suit their snoring behavior and comfort preferences. Customizable settings allow adjustments for light, mild, or loud snorers. The device is suitable for a broad range of users with varying degrees of snoring and apnea severity.

Another object of the SASP is to provide a means to detect snoring. The device responds dynamically to snoring or airway obstruction, adapting to changing conditions throughout the night.

Another object of the SASP is to provide a means to learn a user's sleeping patterns. The snoring detection module improves over time for increased accuracy.

Another object of the SASP is to provide seamless Integration with existing bedding. The SASP fits within a standard pillow design for easy incorporation into existing sleep habits. The SASP blends into a standard bedroom setup without drawing attention and avoids the noise issues commonly associated with CPAP machines

Another object of the SASP is to provide users with a simple operating interface. The SASP includes a mobile app and requires minimal user intervention. The SASP can receive upgrades remotely, ensuring long-term utility without additional hardware investment.

Another object of the SASP is to provide portability. The device is lightweight and compact, making it easy to use at home or while traveling.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting. As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The objects features, and advantages of the present invention, as well as the advantages thereof over existing prior art, which will become apparent from the description to follow, are accomplished by the improvements described in this specification and hereinafter described in the following detailed description which fully discloses the invention.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a top perspective view of the SASP.

FIG. 2 shows top view of the SASP being used.

FIG. 3 shows a top view of the SASP angles.

FIG. 4 shows a side view of the SASP in use.

FIG. 5 shows a flow diagram of the SASP systems.

FIG. 6 shows a representative view of the SASP method.

FIG. 7 shows a perspective view of the SASP.

FIG. 8 shows a top view of the SASP.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure.

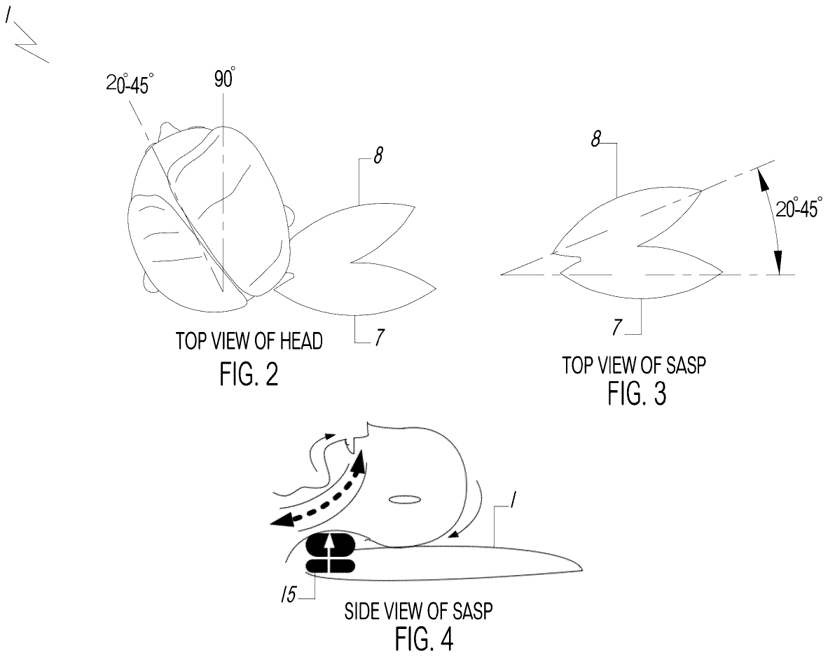

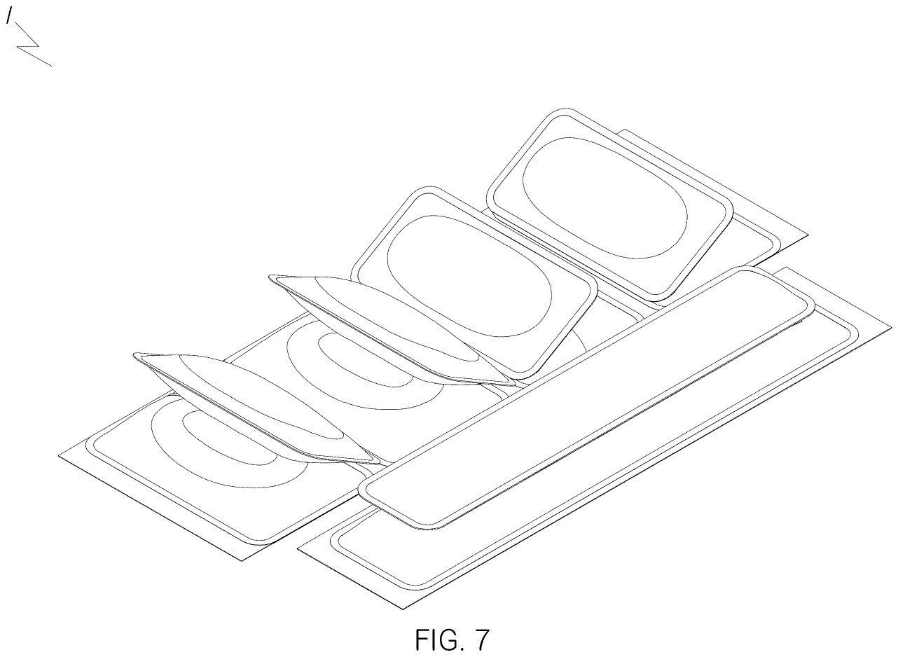

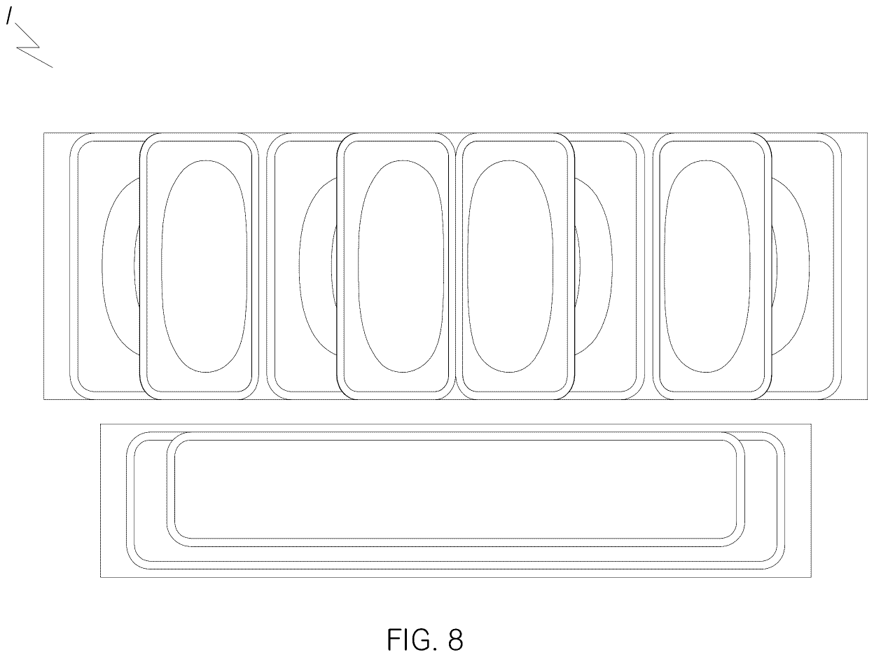

The figures show a preferred embodiment of the SASP 1 being an advanced, intelligently designed device that offers a comprehensive solution to snoring and sleep apnea by dynamically adjusting the head and neck positions to improve airway alignment and enhance sleep quality (as shown in FIGS. 2, 7, 8). The SASP integrates ergonomic design, sophisticated materials, and automated control technologies to deliver a seamless, user-centric experience.

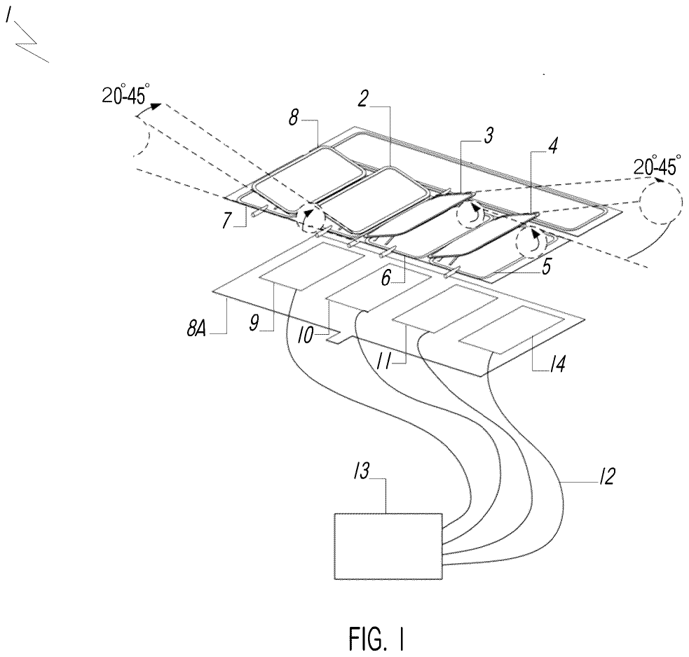

FIG. 1 shows the SASP 1 having base layer 8A connected with a multitude of inflatable, centrally-connected, square-shaped, butterfly side-push air bags 2-8 driven by means of a conventional air pump with onboard microcontroller 13. The base layer 8A of the SASP 1, made from but not limited to a high-density cold foam, provides firm support to maintain butterfly side-push air bag 2-8 structural integrity, ensuring they remain stable even during continuous dynamic adjustments. This feature is particularly crucial for the long-term durability of the embedded mechanisms. Within said base layer 8A sensors including but not limited to audio and vibration sensors are embedded in square-shaped pockets 9-11 and 14 that are connected to the aforementioned microcontroller in pump 13. Said sensors measuring snoring and conditions along the entire SASP 1 area beneath a user's head (to be discussed further in this section). When inflated, the butterfly side-push air bags 2-8 push the head from one side to the other as needed (shown in FIG. 2). The sensor system is comprised of advanced sensors such as but not limited to: audio microphones, vibration sensors; pressure sensors; and position sensors that enable the SASP 1's microcontroller to monitor the user's head posture and allowing microcontroller in air pump 13 to make real-time head angle adjustments by activating the air pump 13 and regulating the pressure supplied to the SASP automatically throughout the sleep period. The microcontroller software includes artificial intelligence and machine learning algorithms that play a pivotal role in coordinating these dynamic adjustments by controlling the air pump 13, pressure sensors, and other critical components. Integrated pressure sensors continuously monitor airbag inflation levels, ensuring that the pressure remains optimal for effective adjustments without risking overinflation. The microcontroller also regulates the timing and intensity of the butterfly side-push air bags 2-8 and horizontal air bag 15 movements, balancing responsiveness with comfort. A wake-on-noise feature further enhances the system's capabilities by ensuring adjustments are initiated promptly upon detecting snoring or other relevant audio signals.

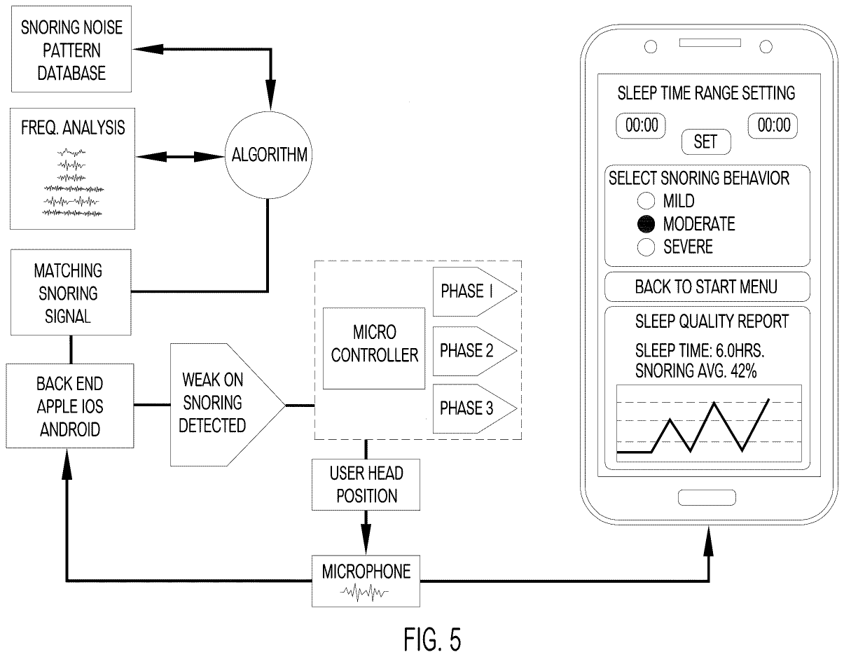

A key differentiator of the SASP form the prior art is its seamless integration with a mobile device software application, which serves as the primary interface for customization, control, and monitoring as shown in FIG. 5. The app connects to the pillow via Bluetooth and provides users with a wide range of options to tailor the device to their specific needs. Users can adjust snoring sensitivity settings, select from light, mild, or loud snorer profiles, and set preferences for the timing and intensity of interventions. The app also offers a manual override feature, enabling users to directly control airbag movements for personalized comfort. Additionally, the app collects and analyzes historical sleep data, generating personalized insights and recommendations to optimize future interventions. The firmware and software are designed to support remote updates, allowing the system to incorporate new features and improvements seamlessly over time.

The middle layer of the SASP 1 is composed of soft material such as memory foam and the like, which is engineered to conform to the contours of the user's head and neck. This viscoelastic material provides personalized comfort by redistributing weight evenly, ensuring the user remains comfortable regardless of the adjustments taking place beneath the surface. A top layer of SASP 1, which is in direct contact with the sleeper, consists of a composite of polyester hollow fiber and cotton. This breathable and soft material ensures a comfortable sleep experience by wicking moisture, promoting airflow, and maintaining a cool surface temperature throughout the night. Together, these layers not only create a luxurious sleeping surface but also ensure the seamless operation of the embedded mechanisms without compromising user comfort.

The innovative airbag system within the SASP 1 is the heart of its functionality. This system comprises two distinct airbag configurations: horizontal neck airbags 15 and the aforementioned butterfly side-push airbags 2-8. The horizontal neck airbags 15 are strategically positioned under the neck region and gently inflate to lift the neck vertically, a motion designed to align the trachea and reduce airway resistance. This vertical adjustment is the first phase or step in SASP's anti-snoring sequence, offering a simple yet effective method to alleviate mild snoring by improving airflow as shown in FIG. 4. Complementing these horizontal neck bags 15 are the butterfly side-push airbags 2-8, symmetrically placed on either side of SASP 1 also shown in FIG. 4. These butterfly side-push airbags 2-8 are designed to perform lateral adjustments, tilting the head incrementally at angles between but not limited to 30° and 45°. This lateral movement facilitates the opening of the airway, particularly in cases of persistent snoring, by repositioning the head to reduce obstructions. All airbags 15 and 2-8 are constructed from medical-grade, highly durable and flexible materials (such as but not limited to a flexible vinyl, plastic and the like), ensuring they remain effective and safe even after prolonged use and repeated inflation cycles.

The functionality of the SASP 1 is driven by an intelligent, multi-phase adjustment mechanism that leverages advanced snoring detection and dynamic response systems. The SASP 1's snoring detection system includes a microcontroller connected to an array of integrated sensors (vibration sensors; pressure sensors; and position sensors etc.) to monitor user's snoring during sleep. These inputs are processed by advanced audio processing software onboard the microcontroller, artificial algorithms and a machine learning module, which can distinguish snoring patterns from ambient noise with a 98% accuracy rate. Over time, the self-learning algorithm refines its detection capabilities by analyzing user-specific snoring behaviors, adapting to unique sleep environments and personal habits. When the system detects snoring, it activates a series of adjustments orchestrated by the embedded microcontroller.

The head adjustment method is designed to address snoring progressively and minimally disturb the sleeper. In the first step (herein referred to as a phase), the horizontal neck airbags 15 inflate gently to elevate the neck, aligning the trachea and opening the airway. If snoring persists, the system moves to the second phase, activating the butterfly side-push airbags 2-8 to tilt the head laterally to at least a 20° angle. For persistent or more severe snoring, the SASP escalates to the third phase, further increasing the lateral adjustment to a 45° angle. This stepwise approach ensures that the system responds precisely to the severity of the snoring while prioritizing the sleeper's comfort. Each inflation cycle is gradual, with the system taking approximately 10 seconds to inflate or deflate, minimizing disruptions to the user's sleep.

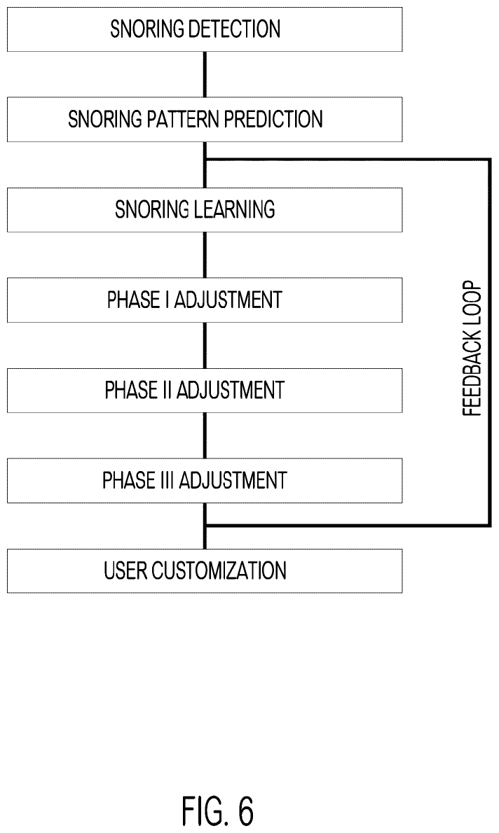

As illustrated in FIG. 6, the SASP 1 incorporates a systematic method to prevent snoring by combining advanced detection, dynamic adjustments, and continuous monitoring. The method steps begin with snoring detection, where integrated microphones and vibration sensors monitor audio and physical signals. These inputs are analyzed by SASP 1's audio processing, artificial intelligence algorithms and machine learning module, which predicts snoring patterns with 98% accuracy while distinguishing them from ambient noise (over time, the SASP 1's self-learning capability refines its detection accuracy by adapting to user-specific snoring behaviors and environmental noise levels). Once snoring is detected, the SASP 1 initiates the next step (a three-phase adjustment sequence). In Phase 1, horizontal neck airbags inflate gently to elevate the neck, aligning the trachea to reduce airway resistance and improve airflow. If snoring detection persists, Phase 2 activates butterfly side-push airbags that tilt the head laterally at a 20° angle, facilitating better airflow by repositioning the head. For continued or severe snoring, Phase 3 increases the lateral adjustment to a 45° angle, maximizing airway opening and minimizing obstructions.

Throughout these phases, adjustments are executed with gradual and controlled inflation and deflation, taking approximately 10 seconds per cycle to minimize disturbances to the sleeper. The system continuously monitors its effectiveness through a feedback loop, pausing interventions if snoring ceases and reinitiating them if snoring resumes. Additionally, the SASP includes a wake-on-noise feature, which activates adjustments promptly upon detecting relevant sounds, ensuring continuous efficacy. Users can customize snoring sensitivity, response thresholds, and intervention timing through an integrated mobile application, which also analyzes historical sleep data to refine future responses. The system's self-learning algorithm further enhances long-term effectiveness by adapting to evolving patterns.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the scope of the invention.

What is claimed is:

1. A system for addressing snoring and improving sleep quality, the system comprising:
a) A pillow structure comprising:
i. A base layer made from a material providing structural integrity;
ii. A plurality of inflatable butterfly side-push airbags symmetrically positioned on the pillow, configured to tilt the head laterally at angles between 20° and 45°;
iii. At least one horizontal neck airbag positioned beneath the neck, configured to elevate the neck vertically to align the trachea;
iv. A soft middle layer composed of viscoelastic material, conforming to the contours of the user's head and neck;
v. A breathable top layer made from a composite material to ensure comfort and airflow;
b) A microcontroller integrated into the system, configured to:
i. Receive input from sensors embedded within the pillow, the sensors including at least audio microphones, vibration sensors, pressure sensors, and position sensors;
ii. Process data from the sensors using onboard audio processing software, artificial intelligence algorithms, and a machine learning module to detect snoring patterns with an accuracy of at least 98%;
iii. Activate an air pump to inflate or deflate the butterfly side-push airbags and the horizontal neck airbags based on detected snoring patterns;
iv. Regulate airbag pressure to maintain optimal inflation levels and prevent overinflation;
v. Execute a three-phase adjustment sequence, wherein: Phase 1 inflates the horizontal neck airbag to elevate the neck vertically; Phase 2 inflates the butterfly side-push airbags to tilt the head laterally at a 20° angle; and Phase 3 adjusts the lateral tilt to a 45° angle if snoring persists;
c) A wake-on-noise feature integrated into the microcontroller, configured to activate adjustments upon detecting relevant sounds; and
d) A mobile application configured to communicate with the microcontroller via Bluetooth, wherein the application:
i. Allows customization of snoring sensitivity settings and intervention timing;
ii. Provides manual control of airbag movements for personalized comfort;
iii. Analyzes historical sleep data to generate personalized recommendations; and
iv. Supports remote firmware updates for system enhancements.

2. A non-transitory computer-readable medium storing instructions executable by a processor to address snoring and improve sleep quality, the instructions comprising the following steps:
a) Providing the system of claim 1;
b) Receiving data from a plurality of sensors embedded within a pillow structure, the sensors including at least audio microphones, vibration sensors, pressure sensors, and position sensors;
c) Processing the sensor data using audio processing algorithms, artificial intelligence algorithms, and a machine learning module to identify snoring patterns with an accuracy of at least 98%;
d) Activating an air pump to dynamically inflate or deflate a plurality of inflatable butterfly side-push airbags and at least one horizontal neck airbag based on detected snoring patterns, wherein: i. The butterfly side-push airbags tilt the user's head laterally at angles between 20° and 45°; ii. The horizontal neck airbag elevates the user's neck vertically to align the trachea;
e) Regulating the timing and pressure of airbag inflation to ensure gradual adjustments and prevent overinflation;
f) Executing a three-phase adjustment sequence to progressively address snoring, wherein: i. Phase 1 inflates the horizontal neck airbag to elevate the neck vertically;
ii. Phase 2 inflates the butterfly side-push airbags to tilt the head laterally at a 20° angle;
iii. Phase 3 increases the lateral tilt to a 45° angle if snoring persists;
g) Activating a wake-on-noise feature to initiate adjustments upon detecting snoring-related sounds; and
h) Communicating with a mobile application via Bluetooth, wherein the application:
allows customization of snoring sensitivity settings and manual control of airbag movements; analyzes historical sleep data to refine future interventions; and
supports updates to the stored instructions for system enhancements.

3. The system of claim 1, wherein the butterfly side-push airbags and horizontal neck airbags are constructed from medical-grade materials, ensuring durability and flexibility during prolonged use.

4. The system of claim 1, wherein the sensors are embedded within square-shaped pockets in the base layer of the pillow.

5. The system of claim 1, wherein the machine learning module adapts to user-specific snoring behaviors over time to improve detection accuracy.

6. The system of claim 1, wherein the mobile application generates personalized sleep profiles based on analyzed historical data.

7. The medium of claim 2, wherein the instructions include enabling time-of-day sensitivity adjustments to reduce intervention thresholds during light sleep stages.

8. The medium of claim 2, wherein the system pauses airbag adjustments when snoring ceases and resumes interventions upon subsequent detection of snoring patterns.

9. The medium of claim 2, wherein updates to the stored instructions include the addition of new features to enhance snoring detection and intervention strategies.

10. The system of claim 1, wherein the breathable top layer wicks moisture and promotes airflow for user comfort.
